(19) United States
(12) Patent Application Publication
(10) Pub. No.: US 2026/0026890 A1
(43) Pub. Date: Jan. 29, 2026

(54) METHOD AND SYSTEM FOR DETERMINING A POSITION OF A CANNULA TIP

(71) Applicant: B. Braun Melsungen AG, Melsungen (DE)

(72) Inventor: Jens Wildhagen, Hannover (DE)

(21) Appl. No.: 19/274,823

(22) Filed: Jul. 21, 2025

(30) Foreign Application Priority Data
Jul. 23, 2024 (DE) 10 2024 120 903.8

(51) Int. Cl. A61B 34/20; A61B 8/08

(52) U.S. Cl. CPC A61B 34/20; A61B 8/0841; A61B 2034/2063

(57) ABSTRACT

A method for determining a position of a cannula tip inside a body includes coupling an ultrasonic signal into the body using an ultrasound probe resting on the body and along an imaging plane. The imaging plane extends along a depth direction and a transverse direction. The ultrasonic signal is received by a plurality of sensors attached to a cannula located in the body and arranged at known axial distances to the cannula tip and to each other along a longitudinal axis of the cannula. The received ultrasonic signals are compared with regard to their signal amplitudes and their signal waveforms by an evaluation device connected to the sensors. The position of the catheter tip relative to the ultrasound probe is determined by the evaluation device depending on the comparison of the signal amplitudes and signal waveforms. A medical system is configured for carrying out the method.

### CROSS-REFERENCE TO RELATED APPLICATION

This application claims priority under 35 U.S.C. § 119 to German Application No. 10 2024 120 903.8, filed on Jul. 23, 2024, the content of which is incorporated by reference herein in its entirety.

### FIELD

The present disclosure relates to a method and a system for determining a position of a cannula tip inside a body.

### BACKGROUND

Such procedures and systems are used, among other things, in the field of regional anaesthesia. From the prior art, a system marked under the registered trademark ONVISION® is known, which is designed to display the position of the cannula tip in an image plane of an ultrasound image. The known system is fully integrated into an underlying ultrasound imaging system.

### SUMMARY

It is an object of the present disclosure to provide a method and a system of the type mentioned above, which offer advantages over the prior art.

The method according to the present disclosure is intended for determining a position of a cannula tip inside a body and comprises the following steps: coupling an ultrasonic signal into the body, wherein the ultrasonic signal is coupled into the body with an ultrasound probe resting on the body and along an imaging plane of an ultrasound imaging procedure, wherein the imaging plane extends along a depth direction and a transverse direction; receiving the ultrasonic signal, wherein the ultrasonic signal is received by a plurality of sensors, which are attached to a cannula located in the body and arranged at known axial distances to the cannula tip and to each other along a longitudinal axis of the cannula; comparing the received ultrasonic signals, wherein the received ultrasonic signals are compared with regard to their signal amplitudes and signal waveforms by means of an evaluation device which is connected to the sensors; optionally comparing the signal amplitudes of the received ultrasonic signals with a signal amplitude of the ultrasonic signal applied to the ultrasound probe; determining the position of the catheter tip in relation to the ultrasound probe, wherein the position is determined by the evaluation device depending on the comparison of the signal amplitudes and the signal waveforms of the received ultrasonic signals, and optionally depending on the comparison of the received signal amplitudes and the transmitted signal amplitudes. The method according to the present disclosure has the advantage that no integration into the ultrasound imaging procedure is required. The method according to the present disclosure is therefore universally applicable. The signal amplitude applied to the ultrasound probe, i.e. the transmitted signal amplitude, can be stored, for example, as a nominal value in a memory unit of the evaluation device. Consequently, no connection of the ultrasound probe to the evaluation device is required. The method according to the present disclosure allows at least an approximate determination of the position of the catheter tip. In one embodiment, an axial distance between the cannula tip and the imaging plane is determined. In a further embodiment, alternatively or additionally an inclination angle of the cannula in a vertical plane is determined. In a further embodiment, alternatively or additionally a depth distance between the ultrasound probe and the cannula is determined. In a further embodiment, alternatively or additionally an angle of rotation of the cannula in a horizontal plane is determined. On the basis of individual, a plurality or all of the above-mentioned variables, the position of the catheter tip with respect to the ultrasound probe can be at least approximately determined.

In an embodiment, determining the position comprises determining an axial distance between the cannula tip and the imaging plane. The axial distance is determined by means of the evaluation device depending on the comparison of the signal amplitudes of the received ultrasonic signals and the known axial distances between the sensors. By comparing the signal amplitudes of the received ultrasonic signals, it is possible to determine which of the plurality of sensors is closest to the imaging plane. Positioning a sensor directly in the imaging plane is expected to result in a maximum of the received ultrasonic signal. Consequently, the sensor at which, in comparison to the other sensors, a maximum signal amplitude of the received ultrasonic signal is present is closest to the imaging plane. Since the axial distances between the sensors and the sensors of the cannula tip are also known, the comparison can be used as a basis to infer the axial distance between the cannula tip and the imaging plane. The axial distance extends along the longitudinal axis of the cannula.

In a further embodiment, the comparison of the signal amplitudes comprises determining the sensor at which, in comparison to the other sensors, a maximum signal amplitude is received. This determination is also carried out by means of the evaluation device. Alternatively, the sensor at which a minimum signal amplitude is received compared to the other sensors can be determined. The sensor with the minimum signal amplitude will usually have the greatest distance to the imaging plane along the longitudinal axis of the cannula.

In a further embodiment, determining the position comprises determining an inclination angle of the cannula, wherein the inclination angle is projected onto a vertical plane which extends along the depth direction and a longitudinal direction, and wherein the inclination angle is determined by the evaluation device depending on the comparison of the signal waveforms of the received ultrasonic signals and the known axial distances between the sensors. The vertical plane extends along the depth direction of the imaging plane and orthogonally to the imaging plane. The inclination angle is determined depending on the comparison of the signal waveforms of the received ultrasonic signals and the known axial distances between the sensors. For example, a phase shift and/or a propagation time difference between the received signals can be determined on the basis of the comparison of the signal waveforms. This phase shift allows an inference to be drawn about a propagation time difference of the transmitted ultrasonic signal up to the individual sensors. For a given frequency of the ultrasonic signal, depending on the phase shift between the received signals of two or more sensors, their distance away from and/or a distance difference relative to the ultrasound probe can be inferred. Alternatively or additionally, the propagation time difference is evaluated directly. The distances or distance differences and/or the propagation time differences together with the known axial distances between the sensors and the cannula tip allow the inclination angle to be determined, for example, on the basis of simple geometric, specifically trigonometric, relationships.

In a further embodiment, the determination of the inclination angle comprises determining a propagation time difference of the received ultrasonic signals for at least one first sensor and one second sensor of the plurality of sensors, wherein the propagation time difference is determined as a function of the comparison of the signal waveforms of the first sensor and the second sensor. In this embodiment of the present disclosure the inclination angle is determined as a function of the propagation time difference and the known axial distance between the first sensor and the second sensor. The propagation time difference is determined depending on the comparison of the signal waveforms of the two sensors, or even a plurality of sensors. The propagation time difference can be determined, for example, as a function of a phase shift of the ultrasonic signal received by the first sensor and the ultrasonic signal received by the second sensor. Alternatively or in addition, the propagation time from the ultrasound probe to each of the sensors can be measured. For this purpose, the ultrasound imaging system can be connected to the evaluation device. Alternatively, the propagation time between the ultrasound probe and the sensors can be determined by one or more further sensors, which is/are arranged directly on or at least near the ultrasound probe.

In a further embodiment, the method comprises a step of comparing the signal amplitudes of the received ultrasonic signals with a signal amplitude of the ultrasonic signal applied to the ultrasound probe by means of the evaluation device, wherein determining the position further comprises determining a depth distance between the ultrasound probe and the cannula. The depth distance extends along the depth direction in the imaging plane. The depth distance is determined by means of the evaluation device depending on the comparison of the transmitted signal amplitude of the ultrasonic signal and the received signal amplitudes of the ultrasonic signal. The greater the difference between the transmitted signal amplitude of the ultrasonic signal and the signal amplitudes of the received ultrasonic signals, the shorter the depth distance between the cannula and the ultrasound probe. Conversely, the greater the difference between the transmitted signal amplitude and the received signal amplitudes, the greater the depth distance. The ultrasound signal is attenuated within the body. This attenuation causes the signal amplitude to decrease with increasing distance from the ultrasound probe in the depth direction. This embodiment of the present disclosure makes use of this circumstance. The transmitted signal amplitude can be stored as a nominal value in a memory unit of the evaluation device. Alternatively, the transmitted signal amplitude can be measured. Alternatively, the ultrasound probe may be connected to the evaluation device for transmitting the transmitted signal amplitude. In a further embodiment, the depth distance is determined as a function of the propagation times. To this end, the time of transmission at the ultrasound probe must be known. The time of transmission can be determined, for example, by a sensor in the immediate vicinity of the ultrasound probe. Alternatively or additionally, the ultrasound probe may be connected to the evaluation device in order to transmit the time of transmission to the evaluation device.

In a further embodiment, determining the position comprises a comparison of an axial profile of the received signal amplitudes over the length of the cannula against reference data. Axial means along the longitudinal axis of the cannula. The reference data represent at least one profile of the signal amplitude of the ultrasonic signal over the longitudinal direction and thus orthogonal to the depth direction and orthogonal to the transverse direction of the imaging plane. The longitudinal direction should not be confused with the longitudinal axis of the cannula. Determining the position further comprises determining a rotation angle of the cannula. The rotation angle is projected onto a horizontal plane extending along the longitudinal and transverse directions. The horizontal plane is thus orthogonal to the imaging plane and orthogonal to the vertical plane which is relevant to the determination of the inclination angle. The rotation angle in the horizontal plane is determined by means of the evaluation device depending on the comparison between the axial profile of the received signal amplitude and the reference data. This embodiment of the present disclosure is based on the consideration that the signal amplitude of the ultrasonic signal decreases with increasing orthogonal distance from the imaging plane, i.e. along the longitudinal direction. If the rotation angle is 0°, the cannula extends longitudinally in the imaging plane. Assuming that the inclination angle is also 0°, the signal amplitude of the received ultrasonic signals over the length of the cannula (i.e. along the longitudinal axis) will be approximately identical. When rotation occurs in the horizontal plane, the distance from the sensors to the imaging plane will vary along the longitudinal direction. This also changes the profile of the signal amplitude of the received ultrasound signal over the longitudinal axis of the cannula. By means of said comparison between the axial profile of the received signal amplitude and the reference data, which represents at least the profile of the signal amplitude of the ultrasonic signal over the longitudinal direction, an inference can be drawn as to the rotation angle of the cannula in the horizontal plane. The reference data is preferably stored in a memory unit of the evaluation device. In one configuration, the reference data can be selected from a reference dataset containing different data for ultrasound probes with different specifications. The selection can be made by a user. In a further embodiment, the reference data is obtained by a reference measurement. Alternatively or in addition, the reference data can represent the curve of the signal amplitude as a function of the inclination angle and/or the rotation angle.

In a further embodiment, the reference data further represent a profile of the signal amplitude of the ultrasonic signal along the depth direction within the imaging plane, wherein the rotation angle is also determined as a function of the determined inclination angle and/or the determined depth distance. By including the inclination angle and/or the depth distance in the determination of the rotation angle, a more accurate determination of the rotation angle and hence the position of the cannula tip is made possible overall.

The medical system according to the present disclosure is configured for determining a position of a cannula tip in a body. The medical system according to the present disclosure has an optional ultrasound probe, a cannula and an evaluation device. The optional ultrasound probe is configured to couple an ultrasonic signal into the body. The cannula comprises the cannula tip and is fitted with a plurality of sensors. The sensors are each configured to receive the ultrasonic signal. The sensors are arranged at known axial distances from the tip of the cannula and from each other along a longitudinal axis of the cannula. The evaluation device is connected to the sensors. This connection can be wireless or wired. The evaluation device is configured for comparing the received ultrasonic signals, in particular at least with regard to the signal amplitudes and/or the signal waveforms of the received ultrasonic signals. Optionally, the evaluation device is further configured for comparing the signal amplitudes of the received ultrasonic signals and a signal amplitude of the ultrasonic signal present at the ultrasound probe. The evaluation device is further configured for determining the position of the catheter tip in relation to the ultrasound probe depending on the comparison of the signal amplitudes and the signal waveforms of the received signals, and optionally depending on the comparison of the received signal amplitude and the transmitted signal amplitude. In other words: the system is configured for carrying out the method according to the present disclosure and its embodiments. In one embodiment, the medical system comprises the optional ultrasound probe already mentioned. In a further embodiment, the medical system does not have such an ultrasound probe. In this case, the ultrasound probe is not part of the medical system, but instead part of an ultrasound imaging system. Embodiments of the medical system arise from the features of the embodiments of the method according to the present disclosure, in particular with regard to any additional unit of the evaluation device.

In a further embodiment, the evaluation device is further configured for comparing the signal amplitudes of the received ultrasonic signals with a signal amplitude of the ultrasonic signal present at the ultrasound probe, and for determining the position of the catheter tip with respect to the ultrasound probe depending on the comparison of the received signal amplitudes and the transmitted signal amplitude. The signal amplitude applied to the ultrasound probe can also be referred to as the transmitted signal amplitude.

### BRIEF DESCRIPTION OF THE DRAWINGS

Further advantages and features of the present disclosure are derived from the following description of preferred exemplary embodiments of the present disclosure, which are explained by reference to the figures.

FIG. 1 shows, in a schematic block diagram, an embodiment of a method according to the present disclosure for determining a position of a cannula tip inside a body;

FIG. 2 shows, in a schematic block diagram, an embodiment of a medical system according to the present disclosure, which is configured for carrying out the method according to FIG. 1;

FIG. 3 shows, in a schematically simplified illustration, an exemplary use situation of the medical system according to FIG. 2;

FIG. 4 shows a diagram with exemplary signal waveforms, which are evaluated for determining the position of the cannula tip;

FIG. 5 shows a further exemplary use situation of the medical system according to FIG. 2;

FIG. 6 shows a further diagram with an exemplary signal waveform;

FIGS. 7, 8 and 9 show further schematic representations of exemplary use situations of the medical system according to FIG. 2;

FIG. 10 shows a diagram with a further exemplary signal waveform;

FIG. 11 shows, in a schematic representation, a further exemplary use situation of the medical system according to FIG. 2; and

FIG. 12 shows a diagram with further exemplary signal waveforms.

### DETAILED DESCRIPTION

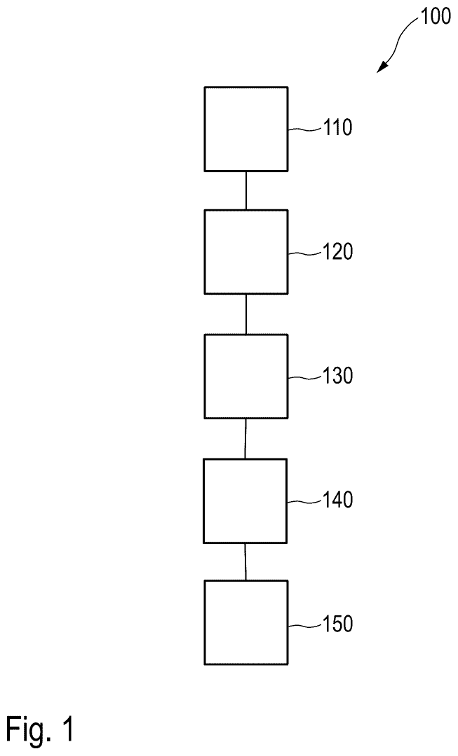

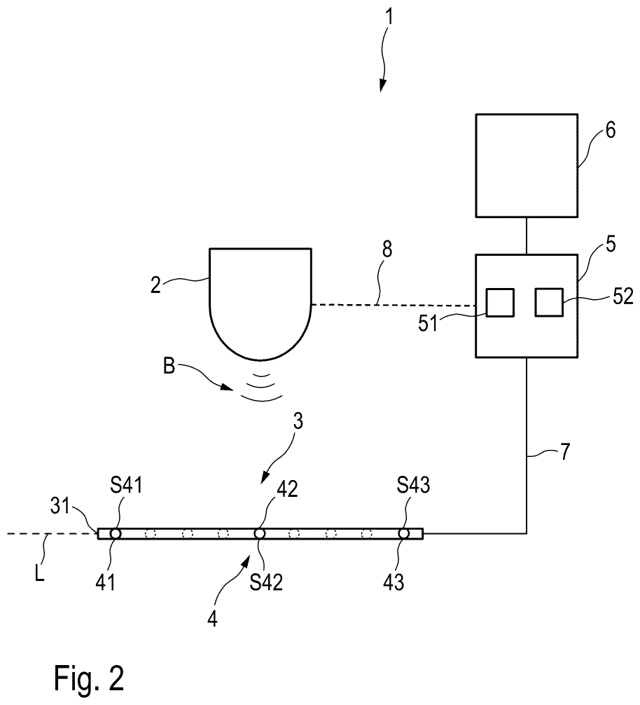

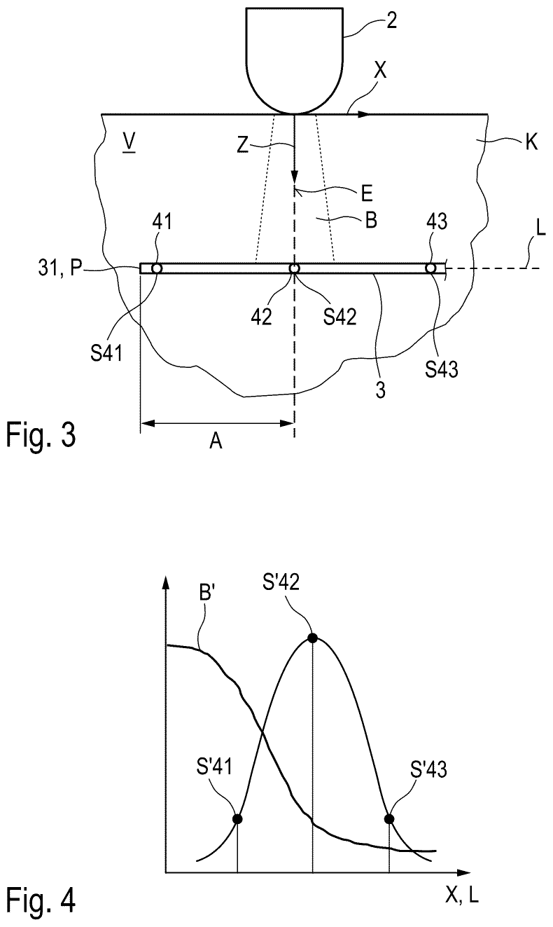

According to FIG. 1, a method 100 for determining a position P of a cannula tip 31 inside a body K (see FIGS. 3, 5, 7, 8, 9, 11) is provided. The method 100 can be carried out with the medical system 1 shown in FIG. 2, in particular in the context of regional anaesthesia. Exemplary use situations of the medical system 1 during the execution of the method 100 are shown in FIGS. 3, 5, 7 to 9 and 11.

The medical system 1 has an optional ultrasound probe 2, a cannula 3, a plurality of sensors 4, an evaluation device 5 and an optional display device 6. The optional ultrasound probe 2 and the optional display device 6 are not present in all embodiments.

Step 110 of the method 100 provides for coupling an ultrasonic signal B into the body K. The ultrasonic signal B is emitted by the ultrasound probe 2 and coupled into the body K. In use, the ultrasound probe 2 rests on the body K. Alternatively, the ultrasound probe 2 may also be inserted into an opening of the body K and arranged inside the body K. The ultrasonic signal B is coupled into the body K along an imaging plane E. The imaging plane E extends along a depth direction Z and a transverse direction Y.

In the embodiment shown in the figures, the ultrasound probe 2 is a component of a separate ultrasound imaging system. The imaging plane E in question is the plane in which an ultrasound image of the ultrasound imaging procedure is generated/displayed. The ultrasonic signal B can also be referred to as a beam when the signal is coupled in. The term “beam” is known to a person skilled in the art.

Step 120 of the method 100 provides for receiving the ultrasonic signal B. The ultrasonic signal B is received using the plurality of sensors 4. The plurality of sensors 4 are attached to the cannula 3 and arranged along a longitudinal axis L of the cannula 3 at known axial distances to the cannula tip 31 and at known distances to each other.

The plurality of sensors 4 are each configured for receiving the ultrasonic signal B and can also be referred to as ultrasonic microphones.

In the embodiment shown, the plurality of sensors 4 comprise a first sensor 41, a second sensor 42 and a third sensor 43. The first sensor 41 is arranged distally in the region of the cannula tip 31. The second sensor 42 is arranged along the longitudinal axis L between the first sensor 41 and the third sensor 43 and can also be referred to as the medial sensor. The third sensor 43 is arranged proximally in the direction of a proximal cannula end of the cannula 3. The proximal cannula end is not shown in this case.

The number of three sensors in the present case is purely exemplary. There may also be more than three sensors present, which is illustrated by the further sensors drawn in dashed lines in FIG. 2 without reference signs.

In exemplary use situations shown, the sensors 41, 42, 43 receive the transmitted ultrasonic signal B. The ultrasonic signals S41, S42, S43 applied to the sensors 41, 42, 43 are also referred to in the following as first ultrasonic signal S41, second ultrasonic signal S42 and third ultrasonic signal S43. The first ultrasonic signal S41 is applied to the first sensor 41. The second ultrasonic signal S42 is applied to the second sensor 42. The third ultrasonic signal S43 is applied to the third sensor 43. The ultrasonic signals S41, S42, S43 differ with regard to their signal amplitude and/or their signal waveforms. These differences are dependent on the respective distance of the relevant sensor 41, 42, 43 from the ultrasound probe 2 and thus from the ultrasound source. Evaluation of the signals S41, S42, S43 thus allows a determination of the position P, wherein the known distances between the sensors 41, 42, 43 and to the cannula tip 31 are also included in the calculation.

The method 100 in step 130 provides a comparison of the received ultrasonic signals S41, S42, S43. This comparison is carried out by means of the evaluation device 5. For this purpose, the evaluation device 5 is connected by means of a signal connection 7 to the sensors 4 attached to the cannula 3. The signal connection 7 is a wired connection in the embodiment shown. In an embodiment not shown in the figures, the signal connection is a wireless connection. The signals S41, S42, S43 received by the sensors 41, 42, 43 are compared with regard to their signal amplitudes and signal waveforms by means of the evaluation device 5. The signal amplitudes of the received signals S41, S42, S43 are marked in the following with a single inverted comma and are consequently referred to as S′41, S′42, S′43.

In step 140 the method 100 provides a comparison of the signal amplitudes S′41, S′42, S′43 of the received ultrasonic signals S41, S42, S43 with a signal amplitude B′ of the ultrasonic signal B applied to the ultrasound probe 2. The signal amplitude B′ of the transmitted ultrasonic signal B is measured in one embodiment. In a further embodiment, the ultrasound probe 2 transmits the signal amplitude B′ to the evaluation device 5, for example via a further signal line 8. This further signal line 8 is optional and therefore shown dashed in FIG. 2. In a further embodiment, the signal amplitude B′ of the coupled-in ultrasonic signal B is given as a nominal value of the ultrasound probe 2. This nominal value can, for example, be input into the evaluation device 5, received from it or stored in it.

The method 100 provides in step 150 a determination of the position P of the catheter tip 31 in relation to the ultrasound probe 2. The position P is determined depending on the comparison of the signal amplitudes S′41, S′42, S′43 and the signal waveforms of the received ultrasonic signals S41, S42, S43 and depending on the comparison of the received signal amplitudes S′41, S′42, S′43 with the transmitted signal amplitude B′ of the ultrasonic signal B. The determination takes place by means of the evaluation device 5.

In the embodiment shown, the evaluation device 5 has a processor unit 51 and a memory unit 52. The processor unit 51 is designed to perform the aforementioned comparisons 130, 140 and the actual determination 150 of the position P. In the embodiment shown, the nominal value of the signal amplitude B′ of the ultrasound probe 2 is stored in the memory unit 52. This nominal value forms a kind of reference value.

The determined position P can be displayed graphically or in a different form by means of the display device 6. For example, the display can be output in relation to the already mentioned imaging plane E. The actual ultrasound image can also be displayed by means of the display device 6.

The display device 6 may be alternatively or additionally designed to provide an acoustically perceptible display or output of the determined position P. For this purpose, the display device 6 may have a loudspeaker unit. The determined position P or its change can in this case be output in a similar way to a Park-Distance-Control in a car, for example with an audio signal sequence, audio signal volume, audio signal level or the like.

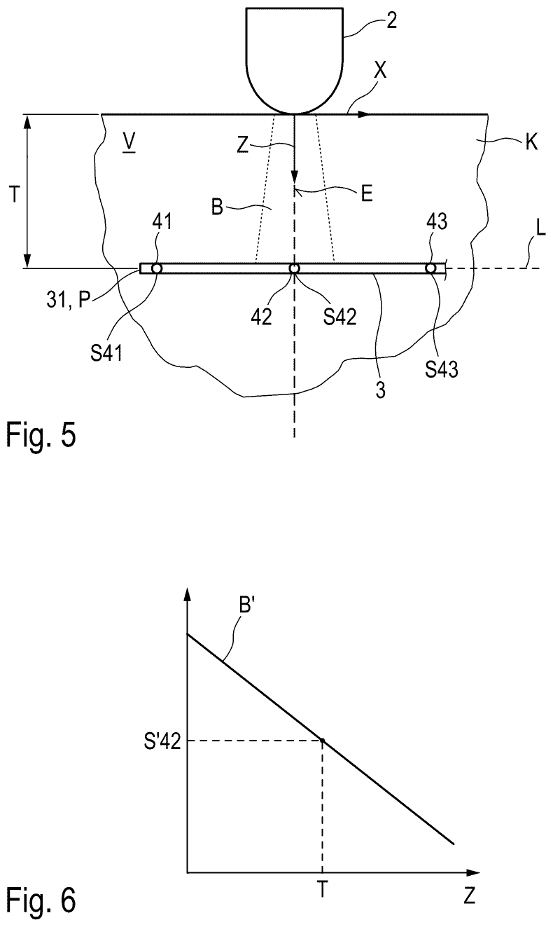

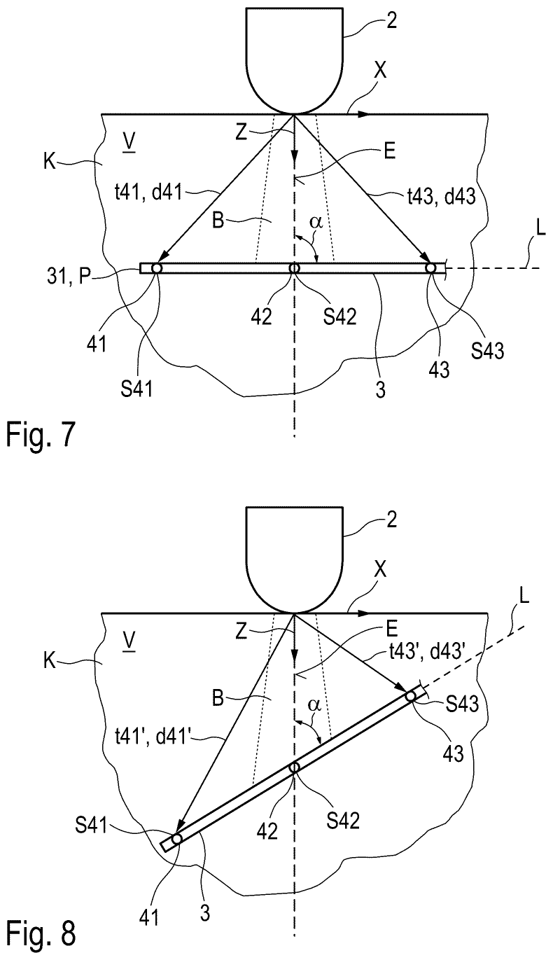

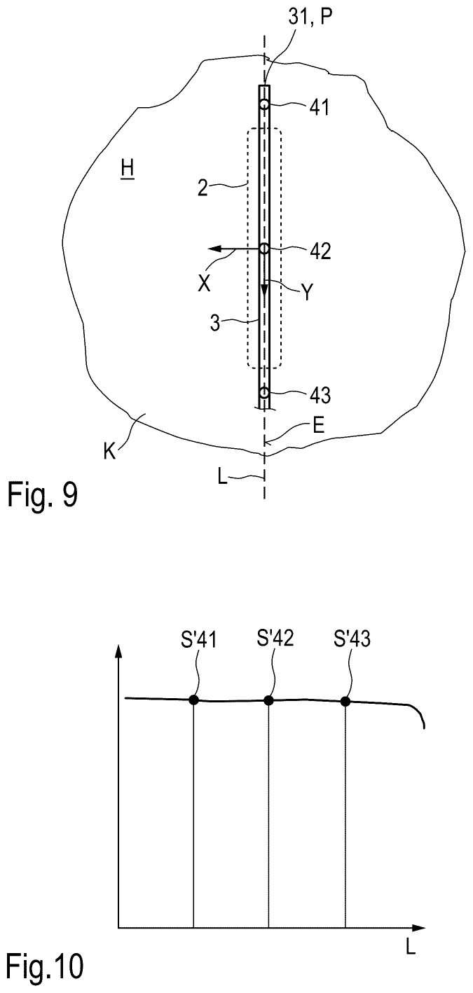

The method 100 and the medical system 1 allow at least an approximate determination of the position P. Approximate means that the position P does not have to be determined uniquely with respect to all coordinate axes. For example, the method 100 and the medical system 1 may be used only to determine an axial distance A (see FIG. 3) between the cannula tip 31 and the imaging plane E. Alternatively or additionally, a depth distance T can be determined (see FIG. 5). As a further alternative or addition, an inclination angle α can be determined (see FIG. 7, 8). As a further alternative or addition, a rotation angle β can be determined (see FIG. 9, 11). Preferably, all of the above-mentioned variables A, T, α, β are determined, so that the position P is determined as accurately and/or unambiguously as possible.

The axial distance A between the cannula tip 31 and the imaging plane E is determined depending on the comparison of the signal amplitudes S′41, S′42, S′43 of the received ultrasonic signals S41, S42, S43 and the known axial distances between the sensors 41, 42, 43 and relative to the cannula tip 31. The determination takes place by means of the evaluation device 5. The determination of the axial distance A is based on the assumption that the sensor positioned the shortest distance away from the imaging plane E delivers a maximum signal amplitude compared to the other sensors. In the exemplary situation shown in FIGS. 3 and 4, the second sensor 42 is positioned directly in the imaging plane E. The second signal amplitude S′42 is therefore a maximum in comparison to the signal amplitudes of the other sensors, in particular the signal amplitudes S′41, S′43. In other words: to determine the axial distance A, the longitudinal position of the cannula 3 at which the beam B is positioned is determined. The axial distance A extends along the longitudinal axis L of the cannula 3. In the exemplary use situation shown in FIG. 3, the longitudinal axis L extends parallel to a longitudinal direction X, which is oriented orthogonal to the depth direction Z and the transverse direction Y of the imaging plane E. In the schematic diagram according to FIG. 4, in addition to the signal amplitudes S′41, S′42, S′43 of the received ultrasonic signals S41, S42, S43, an exemplary profile of the signal amplitude B′ of the (transmitted) ultrasonic signal B over the longitudinal direction X is plotted. As the distance from the imaging plane E increases, the transmitted signal amplitude B′ decreases. This is due to the directivity of the beam B and/or the attenuation resulting from the properties of the body K.

The depth distance T extends in the imaging plane E along the depth direction Z (see FIG. 5). The depth distance T is determined by means of the evaluation device 5 depending on the above-mentioned comparison 140 of the transmitted signal amplitude B′ and the received signal amplitude S′41, S′42, S′43. The determination of the depth distance T is based on the assumption that the signal amplitude B′ of the beam B decreases with increasing depth along the depth direction Z. This decrease is shown in simplified schematic form in the exemplary diagram according to FIG. 6, wherein the exemplary representation is logarithmic. The course of the signal amplitude B′ over the depth direction Z is stored in the embodiment shown in the form of reference data in the memory unit 52 of the evaluation device 5. The reference data can be determined in advance and/or only once, for example, using a reference measurement on a reference measurement setup.

If the profile of the signal amplitude B′ of the transmitted ultrasonic signal B over the depth direction Z is known, a comparison with the received signal amplitudes S′41, S′42, S′43 can be used to infer the depth distance T. For example, if the second sensor 42, as in the exemplary use situation, is located directly in the imaging plane E, the depth distance T between the second sensor 42 and the ultrasound probe 2 along the depth direction Z can be determined by a simple comparison between the second signal amplitude S′42 and the said profile of the signal amplitude B′ over the depth direction Z.

If the cannula 3 were to be inclined at an angle (see, for example, FIG. 8), the depth distance of the cannula tip 31 can be determined via simple trigonometric relationships and on the basis of the known distances between the sensors 41, 42, 43 and the cannula tip 31.

The inclination angle α of the cannula 3 is projected onto a vertical plane V which extends along the depth direction Z and the longitudinal direction X (see FIG. 7, 8). The inclination angle α is determined depending on the comparison of the already mentioned signal waveforms of the received ultrasonic signals S41, S42, S43 and the known axial distances between the sensors 41, 42, 43 and in relation to the cannula tip 31. The inclination angle α is also determined by means of the evaluation device 5.

The determination of the inclination angle α is based on the assumption that different propagation times of the ultrasonic signal B are obtained depending on the distance of the sensors 41, 42, 43 from the ultrasound probe 2. These different propagation times are indicated schematically in FIGS. 7 and 8 for the first sensor 41 and the third sensor 43 respectively, in the form of arrows. The propagation time of the ultrasonic signal B between the ultrasound probe 2 and the first sensor 41 is referred to as t41. The propagation time assigned to the third sensor 43 is designated as t43. In the exemplary use situation according to FIG. 7, the longitudinal axis L is oriented parallel to the longitudinal direction X and the two sensors 41, 43 are positioned at identical axial distances along the longitudinal axis L relative to the imaging plane E. The propagation times t41, t43 are therefore identical. The distances between the ultrasound probe 2 and the two sensors 41, 43 are also identical. These distances are labelled with the reference signs d41, d43 in FIG. 7 and are obtained purely by computation from the propagation time of the transmitted ultrasonic signal B and its frequency. The frequency can be determined, for example, by means of the evaluation device 5 on the basis of the received signals S41, S42, S43. Alternatively or in addition, the frequency of the ultrasonic signal B can be stored in the memory unit 52 of the evaluation device 5 as a nominal value. In the exemplary use situation shown in FIG. 7, there is therefore no phase shift between the signal waveforms of the received ultrasonic signals S41, S43.

The situation is different in the use situation shown in FIG. 8. There, starting from the horizontal orientation according to FIG. 7, the cannula 3 is tilted distally downwards in the depth direction T. The third sensor 43 is therefore closer to the ultrasound probe 2 than the first sensor 41. Consequently, different propagation times t41′, t43′ and thus different distances d41′, d43′ are obtained. In this case the propagation times t41, t43 or t41′, t43′ are not determined by a direct time measurement. Instead, an indirect determination is carried out by comparing the signal waveforms of signals S41, S43 and a resulting phase shift between these two signals S41, S43. The inclination angle α can be determined based on the difference between propagation times and simple trigonometric relationships.

If the inclination angle α is known, the axial distance A and/or the depth distance T can be determined with improved accuracy, and vice versa. This is again based on simple trigonometric relationships and depends on the known distances between the sensors 41, 42, 43 and their distances from the cannula tip 31.

The rotation angle β (see FIG. 9) is an angle projected onto a horizontal plane H. The horizontal plane H is oriented orthogonally to the imaging plane E and the vertical plane V. The horizontal plane H therefore extends along the longitudinal direction X and the transverse direction Y.

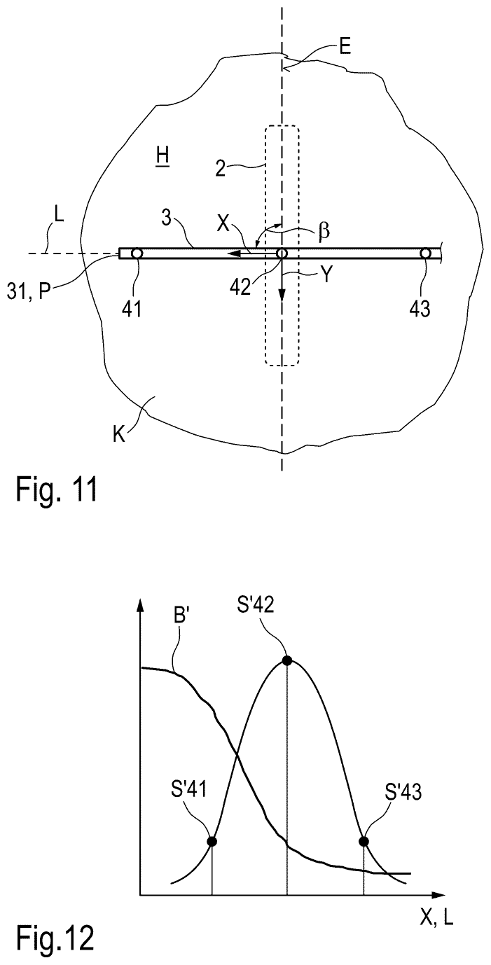

The ultrasound probe 2 is drawn in dashed lines in FIGS. 9 and 11 and extends along the transverse direction Y. The ultrasound probe 2 is elongated with respect to the transverse direction Y. In other words: the dimensions along the transverse direction Y are significantly larger than along the longitudinal direction X.

The determination of the rotation angle β in turn is based on the assumption that the signal amplitude B′ of the coupled-in ultrasonic signal B, i.e. of the beam, decreases with increasing distance from the imaging plane E and thus along the longitudinal direction X. This exemplary decrease of the signal amplitude B′ is shown schematically in FIG. 12.

In the exemplary use situation according to FIG. 9, the rotation angle β is approximately zero and consequently the longitudinal axis L is oriented parallel to the transverse direction Y. Such a longitudinal extension of the cannula 3 within the imaging plane E is also referred to as an arrangement “in plane”. Assuming that the signal amplitude B′ of the beam B in the longitudinal direction Y—at least over the length of the ultrasound probe 2—is constant, an arrangement “in plane” also results in approximately identical signal amplitudes S′41, S′42, S′43 along the longitudinal axis L (see FIG. 10).

As the rotation angle β increases, the difference between the signal amplitudes S′41, S′42, S′43 also increases. This difference is a maximum at a rotation angle β of 90° (see FIGS. 11, 12, arrangement “out of plane”). The rotation angle β can thus be determined by comparing an axial profile of the received signal amplitudes S′41, S′42, S′43 over the length of the cannula 3, i.e. along the longitudinal axis L, with the profile of the signal amplitude B over the longitudinal direction X. The profile of the signal amplitude B′ of the ultrasonic signal B over the longitudinal direction X in the present case is stored in the storage unit 52 in the form of reference data.

1. A method for determining a position of a cannula tip inside a body, the method comprising the steps of:
coupling an ultrasonic signal into the body, wherein the ultrasonic signal is coupled into the body with an ultrasound probe resting on the body and along an imaging plane of an ultrasound imaging procedure, wherein the imaging plane is extended along a depth direction and a transverse direction;
receiving the ultrasonic signal, wherein the ultrasonic signal is received by a plurality of sensors, which are attached to a cannula located in the body and arranged at known axial distances to the cannula tip and to each other along a longitudinal axis of the cannula;
comparing the received ultrasonic signals, wherein the received ultrasonic signals are compared with regard to their signal amplitudes and their signal waveforms by means of an evaluation device which is connected to the sensors; and
determining the position of the cannula tip in relation to the ultrasound probe, wherein the position is determined by the evaluation device depending on a comparison of the signal amplitudes and of the signal waveforms of the received ultrasonic signals.

2. The method according to claim 1, wherein the step of determining the position of the cannula tip further comprises:
determining an axial distance between the cannula tip and the imaging plane, wherein the axial distance is determined by the evaluation device depending on a comparison of the signal amplitudes of the received ultrasonic signals and the known axial distances between the sensors.

3. The method according to claim 2, wherein comparing the comparison of the signal amplitudes comprises determining the sensor at which a maximum signal amplitude is received in comparison to the other sensors.

4. The method according to claim 1, wherein the step of determining the position of the cannula tip further comprises determining an inclination angle of the cannula,
wherein the inclination angle is projected onto a vertical plane that is extended along the depth direction and a longitudinal direction, and
wherein the inclination angle is determined by the evaluation device depending on the comparison of the signal waveforms of the received ultrasonic signals and the known axial distances between the sensors.

5. The method according to claim 4, wherein determining the inclination angle further comprises:
determining a propagation time difference of the received ultrasonic signals for at least a first sensor and a second sensor of the plurality of sensors, wherein the propagation time difference is determined depending on the comparison of the signal waveforms of the first sensor and the second sensor, and
determining the inclination angle as a function of the propagation time difference and the known axial distance between the first sensor and the second sensor.

6. The method according to claim 4, wherein determining the position of the cannula tip comprises the steps of:
comparing an axial profile of the received signal amplitudes over a length of the cannula with reference data representing at least one profile of the signal amplitude of the ultrasonic signal over the longitudinal direction; and
determining a rotation angle of the cannula, wherein the rotation angle is projected onto a horizontal plane which extends along the longitudinal direction and the transverse direction, and wherein the rotation angle is determined by the evaluation device as a function of the comparison between the axial profile of the received signal amplitudes and the reference data.

7. The method according to claim 6, wherein:
the reference data further represent a profile of the signal amplitude of the ultrasonic signal along the depth direction within the imaging plane, and
the rotation angle is determined as a function of the inclination angle and/or the determined depth distance and/or vice versa.

8. The method according to claim 1, further comprising the step of comparing the signal amplitudes of the received ultrasonic signals with a signal amplitude of the ultrasonic signal applied to the ultrasound probe, wherein the comparison is carried out using the evaluation device;
wherein determining the position of the cannula tip further comprises determining a depth distance between the ultrasound probe and the cannula, wherein the depth distance in the imaging plane extends along the depth direction, and wherein the depth distance is determined by the evaluation device depending on the comparison of the signal amplitude applied to the ultrasound probe and the received signal amplitudes.

9. A medical system for determining a position of a cannula tip inside a body, the medical system comprising:
an ultrasound probe configured to couple an ultrasonic signal into the body;
a cannula having the cannula tip and a plurality of sensors configured to receive the ultrasonic signal and arranged at known axial distances to the cannula tip and to each other along a longitudinal axis of the cannula;
an evaluation device that is connected to the sensors and configured to:
compare the received ultrasonic signals, and
determine the position of the cannula tip in relation to the ultrasound probe depending on a comparison of signal amplitudes and signal waveforms of the received ultrasonic signals.

10. The medical system according to claim 9, wherein the evaluation device is further configured to:
compare the signal amplitudes of the received ultrasonic signals with a signal amplitude of the ultrasonic signal applied to the ultrasound probe, and
determine the position of the cannula tip in relation to the ultrasound probe depending on a comparison of the signal amplitudes and the signal amplitude of the ultrasonic signal.
